(19) United States
(12) Patent Application Publication
(10) Pub. No.: US 2009/0274759 A1
(43) Pub. Date: Nov. 5, 2009

(54) SOLID PHARMACEUTICAL COMPOSITION WITH A FIRST FRACTION OF A DISPERSION MEDIUM AND A SECOND FRACTION OF A MATRIX, THE LATTER BEING AT LEAST PARTIALLY FIRST EXPOSED TO GASTROINTESTINAL FLUIDS

(75) Inventors: Daniel Bar-Shalom, Kokkedal (DK); Lillian Slot, Virum (DK)

Correspondence Address: FOLEY AND LARDNER LLP;SUITE 500, 3000 K STREET NW, WASHINGTON, DC, 20007, US

(73) Assignee: Egalet a/s

(21) Appl. No.: 11/915,655

(22) PCT Filed: Jun. 2, 2006
(86) PCT No.: PCT/DK2006/000312

§ 371 Date: Jun. 17, 2008

(30) Foreign Application Priority Data
Jun. 3, 2005 (DK) PA 2005 00813

(51) Int. Cl. A61K 9/14; A61K 38/16

(52) U.S. Cl. 424/484; 514/12

(57) ABSTRACT

A solid pharmaceutical composition in the form of a single dosage unit for oral use, the composition comprising a first and a second fraction, the first fraction comprises a therapeutically and/or prophylactically active substance dispersed in a dispersion medium that is sufficiently fluid at body temperature and the second fraction comprises a matrix comprising a substantially water soluble and/or crystalline polymer or a mixture of substantially water soluble and/or crystalline polymers, the first fraction being included in the composition in such a manner that at least a part of the second fraction is firstly exposed to the gastrointestinal fluids upon administration before the first fraction becomes exposed. The system is designed to release the active substance after a predetermined period of time after administration, and the release of the active substance at that point in time is relatively fast.

### INTRODUCTION

The present invention relates to a drug delivery system for oral administration comprising a diagnostically, therapeutically and/or prophylactically active substance. The system is designed to release the active substance after a predetermined period of time after administration, and the release of the active substance at that point in time is relatively fast.

### BACKGROUND OF THE INVENTION

Many active substances are absorbed in a relatively narrow absorption window in the gastrointestinal tract. Moreover, e.g. with respect to some conditions or diseases such as e.g. arthrosclerosis, many patients suffer from morning stiffness and need proper medication at that time. In such cases, it would be advantageous to administer a pharmaceutical composition that releases the active substance in the morning but that is administered already before going into bed.

Accordingly, there is a need for developing pharmaceutical compositions that are designed to release the active substance after a certain lag time after administration of the composition.

### DESCRIPTION OF THE INVENTION

In one aspect the present invention relates to a solid pharmaceutical composition in the form of a single dosage unit for oral use, the composition comprising a first and a second fraction, the first fraction comprises a therapeutically and/or prophylactically active substance dispersed in a dispersion medium that is sufficiently fluid at body temperature and the second fraction comprises a matrix comprising a substantially water soluble and/or crystalline polymer or a mixture of substantially water soluble and/or crystalline polymers, the first fraction being included in the composition in such a manner that at least a part of the second fraction is firstly exposed to the gastrointestinal fluids upon administration before the first fraction becomes exposed.

A composition according to the invention is typically based on the matrix principle disclosed in e.g. WO 99/51208 (to the same applicant). Such matrices have unique properties in that they erode, i.e. it does not disintegrate into smaller particles or conglomerates of particles upon exposure to the gastrointestinal fluid. Simplified, erosion means that a layer is eroded from the composition into the surrounding medium almost as if a slice of the matrix is cut off. Only the outer surface is exposed to erosion and from this outer layer the active substance will be released and/or dissolved provided that an active substance is present in the matrix. This means that if it is possible to control the size of the surface area by maintaining a constant size, it is possible to control the size of the release rate and, furthermore, a zero order release rate is obtained

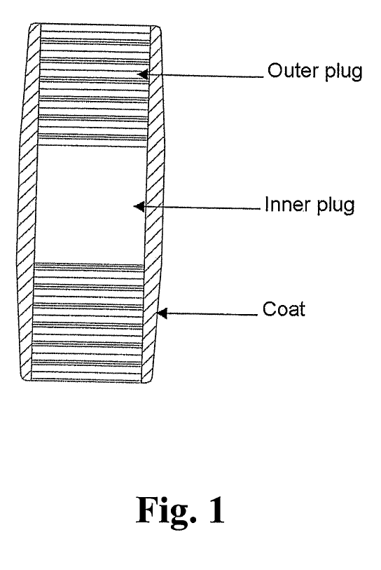

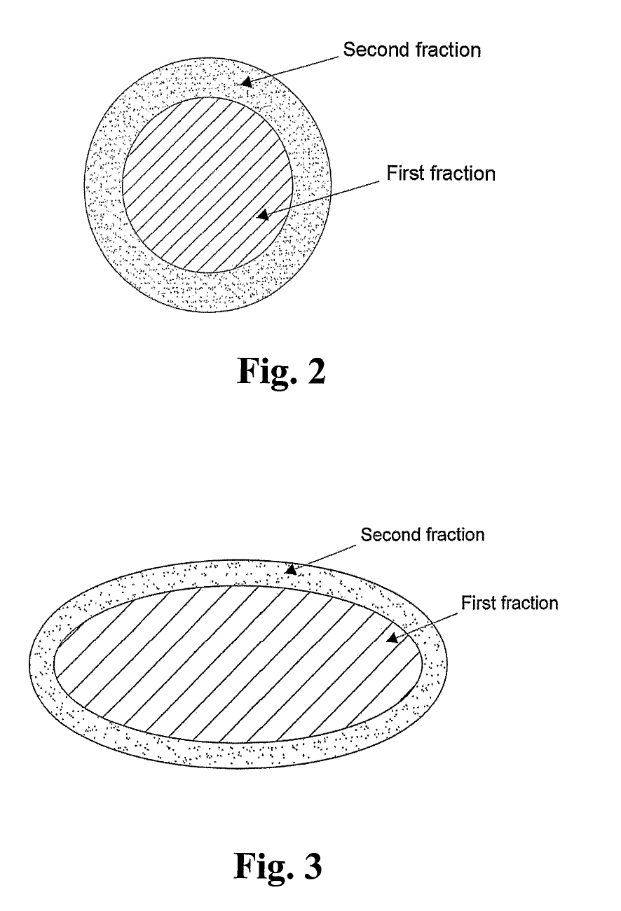

In order to more detailed explain the invention, reference is made to FIG. 1 herein although the invention is not limited to this type and shape of the composition. However, the general idea is to have a layered composition, wherein the first and the second fraction are contained in the composition in the form of separate layers. Thus, the first fraction may be contained in an inner layer of the composition and at least one surface of the inner layer being in contact with at least one surface of the matrix of the second fraction. The most simplified versions of the pharmaceutical composition according to the invention are either a sphere or an oval shaped first fraction surrounded by a sphere or an oval shaped second fraction, as shown in FIG. 2 and FIG. 3, respectively.

In FIG. 1, the first fraction corresponds to an inner plug and the second fraction corresponds to outer plugs (i.e. there are two second fractions). As will be explained later herein, a composition according to the invention may be provided with a coating that can be applied in such a manner that it leaves a well-defined surface area free of coating while the remaining surface is covered and at the same time ensuring that the coating fulfils the requirement that no transport of water into the matrix (or other parts of the composition) takes place through the coating (or, if any water should enter, then it does not result in a transport of dissolved active substance out through the coating). In other words, it has been the object to develop a coating that is not expelled during time (leaving an uncontrollable surface area of the matrix exposed to the aqueous environment) and that has suitable properties compared to the matrix, i.e. if the coating dissolves or otherwise disappears then it should only take place after the matrix has eroded away (during the release period, the coating may of course also partly dissolve or disappear provided that the part it concerns covered a part of the matrix that has already been subject to erosion, thus, leaving the remaining composition “intact” with a matrix that is surrounded by the coating apart from the open end from which erosion of the matrix takes place).

If the coating provided on the composition, if any, is rather rigid it is important to ensure that the first fraction can flow out of the cavity created by erosion of the second fraction. This is obtained by ensuring that the dispersion medium typically is a medium that is in liquid form or semi-solid form at body temperature and if it is in semi-solid form then it has a fluidity that enables it to flow. This is accomplished when the dispersion medium of the first fraction and/or the first fraction itself—when tested according to the Flow Test described herein—passes the test.

Normally, a suitable dispersion medium of the first fraction and/or the first fraction itself has a melting point cut off of at the most about 50° C. In the present context “the melting point cut off” is defined as the temperature that is obtained as the interception with the baseline of the tangent to a DSC curve declining from the peak with increasing temperature to the baseline. In order to ensure that the active substance contained in the first fraction can be suitably released, the end temperature of dispersion medium and/or of the first fraction must be at the most about 50° C. such as, e.g., at the most about 45° C., at the most about 40° C. or at the most about 38° C. In other words, the melting point cut off is determined similarly with the onset temperature, the only difference being that the onset temperature is determined based on the increasing part of the DSC curve whereas the cut off temperature is determined based on the decreasing part of the DSC curve.

The dispersion medium may be liquid even at room temperature or at lower temperatures. This is possible because the first fraction normally is placed between two second fractions, between a coating and a second fraction, or alternatively is completely surrounded by a second fraction. Suitable dispersion medium of the first fraction and/or the first fraction itself may then have a melting point onset of about 0° C. or more such as, e.g. about 5° C. or more, about 10° C. or more, about 15° C. or more, about 20° C. or more or about 25° C. or more.

The dispersion medium normally comprises one or more solvents, one or more co-solvents, one or more oils, one or more waxes and/or one or more semi-solid materials. It may be a lipid-based medium or it may be an aqueous-based medium such as, e.g., an emulsion.

Examples on suitable substances for use in a dispersion medium are lipophilic substances selected from the group consisting of cocoa butter, coca butte substitutes like e.g. vegetable oils modified by esterification, hydrogenation, fractionation etc., shea butter, adeps solidus including those using different triglycerides as starting materials, waxes including beeswax, theobroma oil, hydrogenated vegetable oil bases such as fatty base, wecobee bases, witepsol based water-soluble bases vegetable oil including coconut oil, palm kern oil, cotton seed oil, olive oil, maize oil, peanut oil, sesame oil, sunflower oil, and miglyol 813, and mixtures thereof. Other examples are hydrophilic substances like e.g. polyethylene glycols that have a molecular weight of 20,000 or less, glycerinated gelatines, fatty acid esters of polyethylene glycol.

In specific embodiments of the invention, the first fraction is aqueous based and comprises surface-active agents, emulsifiers and/or nano particles.

The first fraction of a composition according to the invention comprises an active substance. As mentioned above the release of the active substance is delayed because the second fraction (or part of the second fraction) must erode before the first fraction is exposed to the gastrointestinal fluids after oral administration.

Such a delay can be expressed as a requirement with respect to in vitro dissolution. Thus, a composition according to the invention is a composition, wherein—when tested in an in vitro dissolution test—at the most about 5% w/w of the active substance contained in the first fraction is released from the composition within 15 min or more after start of the test.

More specifically, at the most 5% w/w of the active substance contained in the first fraction is released from the composition within 30 min or more such as, e.g., 1 h or more, 1.5 h or more, 2 h or more, 3 h or more, 4 h or more, 5 h or more, 6 h or more, 7 hours or more or 8 hours or more after start of the test.

Alternatively, the release of the active substance from the first fraction—when tested using an in vitro dissolution test method—is at the most about 20% w/w such as at the most about 15% w/w, at the most about 10% w/w, at the most 5% w/w, at the most about 2.5% w/w, at the most about 1% w/w or at the most about 0.1% w/w when measured 2 hours or more such as, e.g., 3 hours or more, 4 hours or more, 5 hours or more, 6 hours or more, 7 hours or more, 8 hours or more, 9 hours or more, 10 hours or more, 11 hours or more, 12 hours or more, 13 hours or more, 14 hours or more, 15 hours or more or 16 hours or more after start of the test.

Details relating to dissolution tests are known to persons skilled in the art of pharmaceutical development such as those defined by the US Pharmacopoeia and Ph.Eur.

Once the first fraction is exposed to the gastrointestinal fluids (or another aqueous medium) the release of the active substance may take place. In contrast to a release from a matrix that erodes (i.e. a matrix like the one of the second fraction), the release of the active substance from the first fraction follows a kinetic that is different from a zero order release. The present formulation principle is designed to delay the release not to enable a zero order release or other types of release kinetics that are relevant when designing controlled or modified release compositions. However, the basic formulation principle of the present invention may be combined with known formulation principles e.g. if an active substance also is included in the matrix of the second fraction, then this active substance can be released by zero order kinetics, i.e. in a controlled manner, and the active substance contained in the first fraction is released in a delayed manner, but once the release from the first fraction starts it may be relatively fast.

Accordingly, a composition according to the present invention may be a composition, wherein at least about 75% w/w such as, e.g., at least about 80% w/w, at least about 85% w/w, at least about 90% w/w or at least about 95% w/w of the total amount of the active substance contained in the first fraction is released within 90 minutes—when tested using an in vitro dissolution test method and the starting point of the test being defined as the point in time when 20% w/w of the total amount of the active substance contained in the first fraction is released.

### Second Fraction—Matrix

The second fraction comprises a matrix. In a specific embodiment the matrix is the second fraction.

### Matrix

The matrix of the second fraction comprises
a) a polymer or a mixture of polymers,
b) optionally, an active substance and,
c) optionally, one or more pharmaceutically acceptable excipients.

In a specific embodiment, the polymer is a substantially water soluble or crystalline polymer or a mixture of substantially water soluble and/or crystalline polymers.

### Polymers

Suitable polymers for use according to the invention typically comprises a polyglycol, e.g. in the form of a homopolymer and/or a copolymer. In a specific embodiment the polymer is substantially water soluble or crystalline polymer or a mixture of substantially water soluble and/or crystalline polymers. Suitable polymers for use in a composition according to the invention are polyethylene oxides and/or block copolymers of ethylene oxide and propylene oxide. Polyethylene oxides which are suitable for use in the matrix composition are those having a molecular weight of from about 20,000 daltons, such as, e.g., from about 20,000 to about 700,000 daltons, from about 20,000 to about 600,000 daltons, from about 35,000 to about 500,000 daltons, from about 35,000 to about 400,000 daltons, from about 35,000 to about 300,000 daltons, from about 50,000 to about 300,000 daltons, such as, e.g. about 35,000 daltons, about 50,000 daltons, about 75,000 daltons, about 100,000 daltons, about 150,000 daltons, about 200,000 daltons, about 250,000 daltons, about 300,000 daltons or about 400,000 daltons.

A particular suitable polyethylene oxide is one, which in itself has a suitable balance between the diffusion rate of water into the polymer and a dissolution rate of the polymer. Suitable examples are polyethylene oxides having a molecular weight of about 35,000 daltons, about 50,000 daltons, about 100,000 daltons, about 200,000 daltons, about 300,000 daltons and about 400,000 daltons.

Poloxamers are copolymers or block copolymers and are a range of non-ionic surfactants of ethylene oxide (EO) and propylene oxide (PO). The composition can be an PO block flanked by polyethylene oxide chain, generating two primary functional hydroxyls or a reversed structure, where a central EO block is sandwiched between a polypropylene glycol group, resulting in an overtone of secondary hydroxyl end groups.

In chemical abstracts Diol EO/PO block copolymers are described under the scientific name—hydroxy-hydroxypoly(oxyethylene)poly(oxypropylene)-poly(oxyethylene)-block copolymer in combination with the CAS register number.

Examples of specific block-copolymers suitable for use in a matrix are:

Poloxamer 101, Poloxamer 105, Poloxamer 108, Poloxamer 123, Poloxamer 124, Poloxamer 181, Poloxamer 182, Poloxamer 184, Poloxamer 185, Poloxamer 188, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407.

Poloxamers are sold under the trademark Pluronic® or Lutrol®.

In specific embodiments a suitable poloxamer for use in a matrix of a composition of the invention has a HLB value of at least about 18 such as, e.g., at least about 20. The mean molecular weight of a suitable poloxamer is typically at least about 2,000. The concentration of the poloxamer in the composition may typically be from about 0% to about 95% w/w such as, e.g., from about 10% to about 90% w/w, from about 10% to about 80% w/w, from about 10% to about 70% w/w, from about 10% to about 60%, from about 10% to about 50%, from about 15% to about 50% w/w, from about 15% to about 45% w/w, from about 15% to about 40% W/W, from about 20% to about 40% w/w, from about 20% to about 35% w/w or from about 20% to about 30% w/w.

Typical block copolymers of ethylene oxide and propylene oxide have a molecular weight of from about 2,000 daltons, typically about 3,000 to about 30,000 daltons such as, e.g. from about 4,000 to about 15,000 daltons.

Polyethylene glycols (which when the molecular weight is above about 20,000 is denoted polyethylene oxides) are mixtures of condensation polymers of ethylene glycol.

Mixtures of PEO with different average molecular weights can be used in order to obtain a PEO with a desirable average molecular weight. It is important to note that in such cases it is necessary to use the PEO, which have MW closest to the desired molecular weight. The individual amount of the two PEO necessary to obtain a PEO with a desired MW can be calculated from the hydroxyl number and the equation given above.

The polymer may have a melting point, which is above the body temperature of the human or animal in which the composition is to be used. Thus, the polymer(s) employed in the matrix composition will suitably have a melting point of about 20-120° C. such as, e.g. from about 30 to about 100° C. or from about 40 to about 80° C.

Alternatively to a polymer of a polyglycol type as described above other polymers may be suitable for use as a) in the matrix composition. Thus, in other embodiments of the invention, the polymer is selected from one or more of the following polymers: water soluble natural polymers such as glucomannan, galactan, glucan, polygalacturonic acid, polyxylane, polygalactomannans, rhanogalacturonan, polyxyloglycan, arabinogalactan, and starch; water soluble polymers such as PVA, PVB, PVP, methocel, Eudragit L methyl ester and PHPV; biodegradable polymers such as PHA, and PLA; hydrogels, such as olyacrylic amid, and dextran; copolymers such as polylactic acid with polyglycolic acid; and others such as alginate and pectins including low methylated or methoxylated pectins.

The concentration of the polymers in the matrix is typically from about 5 to about 99.9% w/w such as from about 10 to about 95% w/w, from about 15% to about 90% w/w, such as from 20 to 85%, such as from 30% to 85% from about 30 to about 99% w/w such as, e.g., from about 35 to about 95% w/w, from about 35 to about 90% w/w, from about 35 to about 85% w/w, from about 35 to about 80% w/w, from about 40 to about 75% w/w, from about 45 to about 70% w/w, from about 45 to about 65% w/w. from about 55 to about 85% w/w or from about 60 to about 85% w/w.

The one or more polymer are typically present in a matrix of a composition of the invention in a concentration amount of from 5 to 99.9% such as from 10 to 95% such as from 15% to 90%, such as from 20 to 85%, such as from 30% to 85% calculated as w/w % of the matrix composition.

The second fraction comprising a matrix typically amount to from about 20% to about 95% w/w such as, e.g., from about 30% to about 90%, from about 40% to about 80%, from about 50% to about 70% or about 60-65% of the (uncoated) composition.

### Active Substances

A composition according to the invention comprises one or more active substances. At least one active substance is included in the first fraction of the composition, but the second fraction may also contain one or more active substances that may be the same or different from the active substance contained in the first fraction.

Typically, the amount of the active substance in the first fraction corresponds to a daily or part of a daily therapeutic dose.

Thus, a pharmaceutical composition according to the invention may comprise one or more active substances, i.e. substances, which are therapeutically, prophylactically, diagnostically and/or biologically active substance. The term “active substance” as used herein broadly includes any compound, or mixture thereof, that can be delivered from the composition to produce a beneficial result. The active and beneficial agents include pesticides, herbicides, germicides, biocides, algicides, rodenticides, fungicides, insecticides, antioxidants, plant hormone promoters, plant growth inhibitors, preservatives, disinfectants, sterilization agents, catalysts, chemical reactants, fermentation agents, food supplements, nutrients, cosmetics, therapeutically active substances (drug substances), vitamins, sex sterilants, fertility inhibitors, fertility promoters, air purifiers, microorganism attenuators, ecological agents and other agents that benefit the environment in which they are used.

In the present context the term “drug substance” includes any physiologically or pharmacologically active substance that produces a localized or systemic effect in animals, in particular in mammals, including humans and primates. Other animals include domestic household, sport or farm animals such as sheep, goats, cattle, horses and pigs, laboratory animals such as mice, rats and guinea pigs, fishes, avians, reptiles and zoo animals. The term “therapeutically, prophylactically and/or diagnostically active substance” includes the term drug substance within its meaning.

In the present context, the term “ecological agent” denotes a non-therapeutic substance that has a biological effect on plants or animals in the environment. An ecological agent may be a pesticide, such as an insecticides or herbicide, a fertilizer a pheromone, a plant growth hormone or the like.

The active substance or substances included in a pharmaceutical composition of the invention may be selected from many therapeutic categories, in particular from substances which may advantageously be administered orally, rectally, vaginally, or administered to a body cavity (e.g. the urinary bladder, kidney pelvis, the gall bladder, the uterus, a central nervous system cavity, infectious/malignant/post-operative cavities, etc.).

Examples of such substances are hypnotics, sedatives, tranquilizers, anti-convulsants, muscle relaxants, analgesics, anti-inflammatory, anaesthetics, anti-spasmodics, anti-ulcer-agents, anti-parasitics, anti-microbials, anti-fungal, cardiovascular agents, diuretics, cytostatics, anti-neoplastic agents, anti-viral agents, anti-glaucoma agents, anti-depressants, sympathomimetics, hypoglycaemics, diagnostic agents, anti-cough, physic energizers, anti-parkinson agents, local anesthetics, muscle contractants, anti-malarials, hormonal agents, contraceptives, anorexic, anti-arthritic, anti-diabetic, anti-hypertensive, anti-pyretic, anti-cholingergic, bronchodilator, central nervous system, inotropic, vasodilator, vasoconstrictor, decongestant, hematine, iron salts and complexes, electrolyte supplement, germicidal, parasympathetolytic, parasympathethomimetic, antiemetic, psychostimulant, vitamin, beta-blockers, H-2 blocker, beta-2 agonist, counterirritants, coagulating modifying agents, stimulants, anti-hormones, drug-antagonists, lipid-regulating agents, uricosurics, cardiac glycosides, ergots and derivatives thereof, expectorants, muscle-relaxants, anti-histamines, purgatives, contrastmaterials, radiopharmaceuticals, imaging agents, anti-allergic agents.

Examples of specific active substances suitable for use in a composition of the invention are:

Carvedilol, morphine, diclofenac, nifedipine, calcitonin, rivastigmine, baclofen, celecoxip, tizanidine, valsartan, telmisartan, losartan, candesartan, eprosartan, irbesartan, galanthamine, methylphenidate, fluoroxetine, rosiglitazone, prednison, prednisolone, codeine, ethylmorphine, dextromethorphan, noscapine, pentoxiverine, acetylcysteine, bromhexine, epinephrine, isoprenaline, orciprenaline, ephedrine, fenoterol, rimiterol, ipratropium, cholinetheophyllinate, proxiphylline, bechlomethasone, budesonide, deslanoside, digoxine, digitoxin, disopyramide, proscillaridin, chinidine, procainamide, mexiletin, flecainide, alprenolol, proproanolol, nadolol, pindolol, oxprenolol, labetalol, timolol, atenolol, pentaeritrityltetranitrate, isosorbiddinitrate, isosorbidmononitrate, niphedipin, phenylamine, verapamil, diltiazem, cyclandelar, nicotinylalcholhol, inositolnicotinate, alprostatdil, etilephrine, prenalterol, dobutamine, dopamine, dihydroergotamine, guanetidine, betanidine, methyldopa, reserpine, guanfacine, trimethaphan, hydralazine, dihydralazine, prazosine, diazoxid, captopril, nifedipine, enalapril, nitroprusside, bendroflumethiazide, hydrochlorthiazide, metychlothiazide, polythiazide, chlorthalidon, cinetazon, clopamide, mefruside, metholazone, bumetanide, ethacrynacide, spironolactone, amiloride, chlofibrate, nicotinic acid, nicheritrol, brompheniramine, cinnarizine, dexchlorpheniramine, clemastine, antazoline, cyproheptadine, proethazine, cimetidine, ranitidine, sucralfat, papaverine, moxaverine, atropin, butylscopolamin, emepron, glucopyrron, hyoscyamine, mepensolar, methylscopolamine, oxiphencyclimine, probanteline, terodilin, sennaglycosides, sagradaextract, dantron, bisachodyl, sodiumpicosulfat, etulos, diphenolxylate, loperamide, salazosulfapyridine, pyrvin, mebendazol, dimeticon, ferrofumarate, ferrosuccinate, ferritetrasemisodium, cyanochobalamine, folid acid heparin, heparin co-factor, diculmarole, warfarin, streptokinase, urokinase, factor VIII, factor IX, vitamin K, thiopeta, busulfan, chlorambucil, cyclophosphamid, melfalan, carmustin, mercatopurin, thioguanin, azathioprin, cytarabin, vinblastin, vinchristin, vindesin, procarbazine, dacarbazine, lomustin, estramustin, teniposide, etoposide, cisplatin, amsachrin, aminogluthetimid, phosphestrol, medroxiprogresterone, hydroxiprogesterone, megesterol, noretisteron, tamoxiphen, ciclosporin, sulfosomidine, bensylpenicillin, phenoxymethylpenicillin, dicloxacillin, cloxacillin, flucoxacillin, ampicillin, amoxicillin, pivampicillin, bacampicillin, piperacillin, meziocillin, mecillinam, pivmecillinam, cephalotin, cephalexin, cephradin, cephadroxil, cephaclor, cefuroxim, cefotaxim, ceftazidim, cefoxitin, aztreonam, imipenem, cilastatin, tetracycline, lymecycline, demeclocycline, metacycline, oxitetracycline, doxycycline, chloramphenicol, spiramycin, fusidic acid, lincomycin, clindamycin, spectinomycin, rifampicin, amphotericin B, griseofulvin, nystatin, vancomycin, metronidazole, tinidazole, trimethoprim, norfloxacin, salazosulfapyridin, aminosalyl, isoniazid, etambutol, nitrofurantoin, nalidixic acid, metanamine, chloroquin, hydroxichloroquin, tinidazol, ketokonazol, acyclovir, interferon idoxuridin, retinal, tiamin, dexpantenol, pyridoxin, folic acid, ascorbic acid, tokoferol, phytominadion, phenfluramin, corticotropin, tetracosactid, tyrotropin, somatotoprin, somatrem, vasopressin, lypressin, desmopressin, oxytocin, chloriongonadotropin, cortison, hydrocortisone, fluodrocortison, prednison, prednisolon, fluoximesteron, mesterolon, nandrolon, stanozolol, oximetolon, cyproteron, levotyroxin, liotyronin, propylthiouracil, carbimazol, tiamazol, dihydrotachysterol, alfacalcidol, calcitirol, insulin, tolbutamid, chlorpropamid, tolazamid, glipizid, glibenclamid, phenobarbital, methyprylon, pyrityldion, meprobamat, chlordiazepoxid, diazepam, nitrazepam, oxazepam, dikaliumclorazepat, lorazepam, flunitrazepam, alprazolam, midazolam, hydroxizin, chlometiazol, propionmazine, alimemazine, chlorpromazine, levomepromazine, acetophenazine, fluphenazine, perphenazine, prochlorperazine, trifluoperazine, dixyrazine, thiodirazine, periciazin, chloprothixene, zuclopentizol, flupentizol, thithixen, haloperidol, trimipramin, opipramol, chlomipramin, desipramin, lofepramin, amitriptylin, nortriptylin, protriptylin, maptrotilin, caffeine, cinnarizine, cyclizine, dimenhydinate, meclozine, prometazine, thiethylperazine, metoclopramide, scopolamine, phenobarbital, phenytoine, ethosuximide, primidone, carbamazepine, chlonazepam, orphenadrine, atropine, bensatropine, biperiden, metixene, procylidine, levodopa, bromocriptin, amantadine, ambenon, pyridostigmine, synstigmine, disulfiram, morphine, codeine, pentazocine, buprenorphine, pethidine, phenoperidine, phentanyl, methadone, piritramide, dextropropoxyphene, ketobemidone, acetylsalicylic acid, phenazone, phenylbutazone, azapropazone, piroxicam, ergotamine, dihydroergotamine, cyproheptadine, pizitifen, flumedroxon, allopurinol, probenecid, sodiummaurothiomalate auronofin, penicillamine, estradiol, estradiolvalerianate, estriol, ethinylestradiol, dihydrogesteron, lynestrenol, medroxiprogresterone, noretisterone, cyclophenile, clomiphene, levonorgestrel, mestranol, ornidazol, tinidazol, ekonazol, chlotrimazol, natamycine, miconazole, sulbentin, methylergotamine, dinoprost, dinoproston, gemeprost, bromocriptine, phenylpropanolamine, sodiumchromoglicate, azetasolamide, dichlophenamide, betacarotene, naloxone, calciumfolinate, in particular clonidine, thephylline, dipyradamol, hydrochlothiazade, scopolamine, indomethacine, furosemide, potassium chloride, morphine, ibuprofen, salbutamol, terbutalin, sulfonylurea, metformin, insulin, calcitonin, glucagons-like peptide-1, or combinations thereof.

The active substance can be in various forms, such as uncharged or charged molecules, molecular complexes, crystalline forms, amorphous form, polymorphous form, solvates, anhydrates, pharmacologically acceptable salts such as a hydrochloride, hydrobromide, sulfate, laurylate, palmitate, phosphate, nitrite, nitrate, borate, acetate, maleate, tartrate, oleate, and salicylate. For acidic active substance, salts of metals, amines amino acids or organic cations, quaternary ammoniums, can be used. Derivatives of active substances such as esters, ethers and amides which have solubility characteristics suitable for use herein can be used alone or mixed with other drugs. After release of the derivative from the drug delivery system it may be converted by enzymes, hydrolysed by body pH or other metabolic processes to the parent drug or to another biologically active form.

A pharmaceutical composition of the invention may in addition be suitable for the delivery of peptides, polypeptides or proteins, for example hormones, enzymes such as lipases, proteases, carbohydrates, amylases, lactoferrin, lactoperoxidases, lysozymes, nanoparticles, etc., and antibodies. The composition may also be employed for the delivery of microorganisms, either living, attenuated or dead, for example bacteria, e.g. gastrointestinal bacteria such as streptococci, e.g. S. faecium, Bacillus spp. such as B. subtilis and B. licheniformis, lactobacteria, Aspergillus spp., bifidogenic factors, or viruses such as indigenous vira, enterovira, bacteriophages, e.g. as vaccines, and fungi such as baker's yeast, Saccharomyces cerevisiae and fungi imperfecti.

A further use for which a composition of the invention is suited is the delivery of active substances to animals. Examples of such active substances for veterinary use are antiparasitics, corticosteroids, antibiotics, antiinflammatory agents, growth promoters and permittants, antifungals and antihelmintics.

The matrix of the second fraction of a pharmaceutical composition of the invention may be designed to release an active substance, if any, in a controlled manner such as by a zero order release mechanism. Accordingly, the composition is also suitable for controlled release of an active substance, i.e. first a controlled release of an active substance (from the matrix, second fraction) and then a relatively fast release of the same or different active substance (from the first fraction) or other suitable release combinations. In the present context the tern “controlled release” is used to designate a release a desired rate during a predetermined release period. Terms like “modified”, “delayed”, “sustained”, “prolonged”, “extended” etc. release are in the present context synonyms to the term “controlled release”.

In an embodiment of the invention, the active substance is a pharmaceutically active powder. The powder typically has a particle size of from about 0.1 μm to about 500 μm, typically from about 0.5 μm to about 300 μm, more typically from about 1 μm to about 200 μm, especially from about 5 μm to about 100 μm.

A pharmaceutical composition according to the invention is suitable for use for water soluble as well as slightly soluble or insoluble active substances. However, it is contemplated that a composition is especially suitable for use when the at least one therapeutically, prophylactically and/or diagnostically active substance has a solubility of at the most about 3 mg/ml such as, e.g. at the most about 1 mg/ml, at the most about 0.1 mg/ml, at the most about 0.05 mg/ml such as, e.g. at the most about 0.001 mg/ml in water at ambient temperature.

The at least one therapeutically, prophylactically and/or diagnostically active substance will suitably be present in an amount of up to about 80%, typically up to about 70%, up to about 60% or up to about 50%, such as, e.g., from 0.1% to 80%, such as from 0.25% to 75%, such as from 0.5% to 60%, such as from 0.75% to 50%, such as from 1% to 40%, such as from 1.5% to 35%, such as from 1.75% to 30% by weight of the composition or first fraction. With respect to situations where one or more active substances are contained in a second fraction, a content of about 60-80% w/w is contemplated to be the maximum content, which still allows for a sufficient content of the polymer and, when relevant, a pharmaceutically acceptable excipient in the composition. The active substance may, on the other hand, be present in the composition in much smaller amounts, depending on the nature and potency of the active substance in question.

A composition according to the invention containing a drug substance is typically for oral administration. Due to the possibility of controlling the release rate of the active substance the composition may be adapted for oral administration 1-6 times a day, normally 1-4 times daily such as 1-3 times, 1-2 times or 1 times daily. The technology may also provide compositions for administration only once or twice daily. In the present context the term “once daily” is intended to mean that it is only necessary to administer the pharmaceutical composition once a day in order to obtain a suitable therapeutic and/or prophylactic response; however, any administration may comprise co-administration of more than one dosage unit, such as, e.g. 2-4 dosage units if the amount of active substance required may not be formulated in only one composition or if a composition of a smaller size is preferred.

The dosage of the active substance depends on the particular substance, the age, weight condition etc. of the human or animal that will be treated with the composition etc. All such factors are well known to a person skilled in the art.

### Pharmaceutically Acceptable Excipients

A pharmaceutical composition according to the invention may comprise one or more pharmaceutically acceptable excipients. The excipient may be present in the first and/or the second fraction of the composition.

A suitable pharmaceutically acceptable excipient for use in composition of the invention may be selected from the group consisting of fillers, diluents, disintegrants, glidants, pH-adjusting agents, viscosity adjusting agents, solubility increasing or decreasing agents, osmotically active agents and solvents.

Suitable excipients include conventional tablet or capsule excipients. These excipients may be, for example, diluents such as dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol or mixtures thereof; binders such as acacia, sodium alginate, starch, gelatin, saccharides (including glucose, sucrose, dextrose and lactose), molasses, extract of Irish moss, panwar gum, ghatti gum, mucilage of isapol husk, carboxymethylcellulose, methylcellulose, veegum, larch arabolactan, polyethylene glycols, ethylcellulose, water, alcohols, waxes, polyvinylpyrrolidone such as, e.g., PVP K90 (may be used to improve mixing of the polymer with the other ingredients) or mixtures thereof; lubricants such as talc, magnesium stearate, calcium stearate, stearic acid, hydrogenated vegetable oils, sodium benzoate, sodium chloride, leucine, carbowax 4000, magnesium lauryl sulfate, colloidal silicon dioxide and mixtures thereof, disintegrants such as starches, clays, cellulose derivatives including crosscarmellose, gums, aligns, various combinations of hydrogencarbonates with weak acids (e.g. sodium hydrogencarbonate/tartaric acid or citric acid) crosprovidone, sodium starch glycolate, agar, cation exchange resins, citrus pulp, veegum HV, natural sponge, bentonite or mixtures thereof; volatile solvents such as alcohols, including aqueous alcohols, petroleum benzine, acetone, ether or mixtures thereof; plasticizers such as sorbitol and glycerine; and others such as cocoa butter, polyethylene glycols or polyethylene oxides, e.g. with a molecular weight of about 1,000-500,000 daltons, typically about 1,000-100,000 daltons, more typically 1,000-50,000 daltons, especially about 1,000-10,000 daltons, in particular about 1,500-5,000 daltons, and mixtures thereof, hydrogenated vegetable oils, glycerinated gelatin or mixtures thereof.

The matrix composition may in addition include a cellulose derivative, e.g. a cellulose derivative selected from the group consisting of methylcellulose, carboxymethylcellulose and salts thereof, microcrystalline cellulose, ethylhydroxyethylcellulose, ethylmethylcellulose, hydroxyethylcellulose, hydroxyethylmethylcellulose, hydroxypropylcellulose, hydroxypropylmethylcellulose, hydroxymethylcellulose and hydroxymethylpropylcellulose. Of these cellulose derivatives, hydroxypropylmethylcellulose and methylcellulose are preferred for incorporation in the composition.

Furthermore, the matrix composition may comprise one or more agents selected from the group consisting of sweetening agents, flavoring agents and coloring agents, in order to provide an elegant and palatable preparation. Examples of coloring agents are water-soluble FD&C dyes and mixtures thereof with corresponding lakes and direct compression sugars such as Di-Pac from Amstar. In addition, colored dye migration inhibitors such as tragacanth, acacia or attapulgite talc may be added. Specific examples include calcium carbonate, chromium-cobalt-aluminium oxide, ferric ferrocyanide, ferric oxide, iron ammonium citrate, iron (III) oxide hydrated, iron oxides, magnesium carbonate, titanium dioxide.

Examples of suitable fillers are also dextrin, sucralfate, calcium hydroxyl-apatite, calcium phosphates and fatty acid salts such as magnesium stearate.

The filler may be added in an amount so that the combination of the filler and the active substance comprises up to about 60%, typically up to about 50%, by weight of the first composition.

In order to soften the composition, a plasticziser may be incorporated in the composition. A suitable plasticizer is selected from the group consisting of phosphate esters; phthalate esters; amides; mineral oils; fatty acids and esters; fatty alcohols, vegetable oils and hydrogenated vegetable oils including acetylated hydrogenated cottonseed glyceride and acetylated hydrogenated soybean oil glycerides; acetyl tributyl citrate, acetyl triethyl citrate, Castor oil, diacetylated monoglycerides, dipropylene glycol salicylate glycerin, glyceryl cocoate, mono- and di-acetylated monoglycerides, nitrobenzene, carbon disulfide, β-naphtyl salicylate, phthalyl glycolate, diocyl phthalate; sorbitol, sorbitol glyceryl tricitrate; sucrose octaacetate; a-tocopheryl polyethylene glycol succinate, phosphate esters; phthalate esters; amides; mineral oils; fatty acids and esters; fatty alcohols; and vegetable oils, fatty alcohols including cetostearyl alcohol, cetyl alcohol, stearyl alcohol, oleyl alcohol and myristyl alcohol; methyl abietate, acetyl tributyl citrate, acetyl triethyl citrate, diisooctyl adipate, amyl oleate, butyl ricinoleate, benzyl benzoate, butyl and glycol esters of fatty acids, butyl diglycol carbonate. butyl oleate, butyl stearate. di(beta-methoxyethyl) adipate, dibutyl sebacate, dibutyl tartrate, diisobutyl adipate, dihexyl adipate, triethylene glycol di(beta-ethyl butyrate), polyethylene glycol di(2-ethyl hexoate), diethylene glycol monolaurate, monomeric polyethylene ester, hydrogenated methyl ester of rosin, methoxyethyl oleate, butoxyethyl stearate, butyl phthalyl butyl glycolate, glycerol tributyrate, triethylene glycol dipelargonate, beta-(p-tert-amyl phenoxy)ethanol, beta(p-tert-butytphenoxy)ethanol, beta-(p-teft-butytphenoxyethyl)acetate, bis(beta-p-tert-buthylphenoxydiethyl)ether, camphor, Cumar W-1, Cumar MH-1, Cumar V-1, diamyl phthalate, (diamylphenoxy) ethanol, diphenyl oxide, technical hydroabietyl alcohol, beckolin, benzene hexahydrochlonde, Clorafin 40, Piccolastic A-5, Piccalastic A-25, Flexol B-400, Glycerol alfa-methyl alfa-phenyl ether, chlorinated naphthalene, HB-40, monoamylphthalate. Nevillac 10 o-nitrodiphenyl and Paracril 26.

Preferred anti-oxidative agents include TPG e.g. in the form of TPGS due to surfactant properties, BHA, BHT, t-butyl hydroquinone, calcium ascorbate, gallic acid, hydroquinone, maltol, octyl gallate, sodium bisulfite, sodium metabisulfite, tocopherol and derivates thereof, citric acid, tartaric acid, and ascorbic acid. Other antioxidants include trivalent phosphorous like e.g phosphite, phenolic antioxidants, hydroxylamines, lactones such as substituted benzofuranones. Hindered phenols, thiosynergists and/or hindered amines are useful for the long-term stability for polymers, whereas the following antioxidants are suitable for use also in situation where the active substance is subject to oxidation: acids (ascorbic acid, erythorbic acid, etidronic acid, gallic acid, hypophosphorous acid, nordihydroguairetic acid, propionic acid etc.), phenols (e.g. BHA, BHT, t-butyl hydroquinone, dodecyl gallate, octyl gallate, 1,3,5-trihydroxybenzene), organic and inorganic salts (calcium ascorbate, sodium ascorbate, sodium bisulphite, sodium metabisulfite, sodium sulfite, potassium bisulphite, potassium metabisulphite), esteres (calcium ascorbate, dilauryl thiodipropionate, dimyristyl thiodipropionate, distearyl thiodipropionate), pyranon (maltol), and vitamin E (tocopherol, D-α-tocopherol, DL-α-tocopherol, tocopheryl acetate, d-α-tocopheryl acetate, dl-α-tocopheryl acetate. However, other anti-oxidative agents known in the art may be used according to the present invention.

### Coating

As mentioned above, a composition according to the invention may be coated. In those cases where a coating functions to ensure that a controlled surface area of the matrix is exposed to the surrounding body fluid, the coating normally has at least one opening exposing one surface of the matrix of the second fraction. However, in those situations where a composition of the invention does not need a coating, i.e. in those cases where the first fraction is embedded or encapsulated in the second fraction, then the composition may or may not be provided with a coating. In the latter case, such a coating may impart further delayed properties for releasing the active substance or it may be a film-coating or other kinds of coatings that does not delay the release but e.g. make it easier to swallow the composition or it may e.g. mask a bad taste. Materials suitable for such coatings are well-known for a person skilled in the art and information can be found e.g. in the latest editions of handbooks like Handbook of Pharmaceutical Excipients or in Remington's Pharmaceutical Sciences.

As mentioned herein before the pharmaceutical composition may thus have the shape of a cylindrical rod, which may be provided with a coating, which is substantially insoluble in and impermeable to fluids such as body fluids during the intended release period, the coating having an opening at one or both ends. Polymers useful as coatings are preferably those, which are possible to process by extrusion, solution or in the form of a dispersion. Most preferred are those, which are available in a food grade or a pharmaceutical grade quality. Examples of such polymers are cellulose acetate, polyamide, polyethylene, polyethylene terephthalate, polypropylenem polyurethane, polyvinyl acetate, polyvinyl chloride, silicone rubber, latex, polyhydroxybutyrate, polyhydroxyvalerate, teflon, polylactic acid or polyglycolic acid and copolymers thereof, copolymers such as ethylene vinyl acetate (EVA), styrene-butadienestyrene (SBS) and styrene-isoprene-styrene (SIS).

The coating may also be a coating, which is substantially soluble in and permeable to fluids such as body fluids during the intended release period provided that the coating dissolves so much slower than the matrix composition that the coating remains intact until the matrix has eroded and released the active substance. Examples of suitable polymers include polyols as described herein.

The coating may further comprise any of the above-mentioned matrix materials in a form, which erodes at a substantially slower rate than the rest of the matrix. The coating may thus comprise a matrix of one or more substantially water soluble crystalline polymers and, optionally, a non-ionic emulsifier, the coating being one which is eroded in the aqueous phase at a substantially slower rate than the matrix composition comprising the active substance, whereby a substantially constant area of the matrix composition comprising the active substance is exposed during erosion of the matrix composition, and whereby the coating is substantially eroded upon erosion of the matrix composition comprising the active substance. Such a coating will be designed so that its longitudinal erosion rate is substantially the same as the longitudinal erosion rate of the matrix, whereby the matrix and the coating will erode longitudinally towards the centre of the composition at substantially the same rate. Thus, when the matrix composition has been completely eroded by the aqueous medium, the coating will also be substantially completely eroded. A matrix composition having such a coating has the obvious advantage of being completely biodegraded upon release of the active substance. Such a coating will typically be a combination of a polyethylene glycol and a mixture of, for example, polyethylene glycol 400 monostearate or another non-ionic emulsifier, and may also include a filler. The content of the mixture of non-ionic emulsifiers and the filler in the coating will be determined in each particular case according to the characteristics, e.g. erosion rate and size, of the matrix comprising the active substance.

In an embodiment of the invention, the coating is one, which disintegrates or crumbles after erosion of the matrix. A coating of this type will remain intact as long as it is supported by the matrix containing the active substance, but it lacks the ability to remain intact after erosion of the matrix, because it then disintegrates or crumbles, so that it will not remain in e.g. a human or animal for any significant amount of time after the complete erosion of the matrix and the release of the active substance.

The coating may also be an enteric coating employing methacrylates, a co-polymer of methacrylate-galactomannan etc.

In an interesting embodiment, the controlled release composition of the invention further comprises a coating having at least one opening exposing at least one surface of the matrix, the coating being one which crumbles and/or erodes upon exposure to the aqueous medium at a rate which is equal to or slower than the rate at which the matrix erodes in the aqueous medium, allowing exposure of said surface of the matrix to the aqueous medium to be controlled. Coatings of this type are described in WO 95/22962, to which reference is made and which is incorporated herein by reference. These coatings comprise:
(a) a first cellulose derivative which has thermoplastic properties and which is substantially insoluble in the aqueous medium in which the composition is to be used, e.g. an ethylcellulose such as ethylcellulose having an ethoxyl content in the range of 44.5-52.5%, or cellulose acetate, cellulose propionate or cellulose nitrate;
   and at least one of:
(b) a second cellulose derivative which is soluble or dispersible in water, e.g. a cellulose derivative selected from the group consisting of methylcellulose, carboxymethylcellulose and salts thereof, cellulose acetate phthalate, microcrystalline cellulose, ethylhydroxyethylcellulose, ethylmethylcellulose, hydroxyethylcellulose, hydroxyethylmethylcellulose, hydroxypropylcellulose, hydroxypropylmethylcellulose, hydroxymethylcellulose and hydroxymethylpropylcellulose;
(c) a plasticizer, e.g. selected from the group consisting of phosphate esters; phthalate esters; amides; mineral oils; fatty acids and esters thereof with polyethylene glycol, glycerin or sugars; fatty alcohols and ethers thereof with polyethylene glycol, glycerin or sugars; and vegetable oils; or a non-ionic surfactant; and
(d) a filler, e.g. selected from conventional tablet or capsule excipients such as diluents, binders, lubricants and disintegrants.

The first cellulose derivative (a) such as, e.g., ethylcellulose is typically contained in the coating in a concentration of from about 10 to about 99% w/w such as, e.g., from about 20 to about 95% w/w, from about 30 to about 90% w/w, from about 40 to about 90% w/w, from about 45 to about 90% w/w, from about 50 to about 85% w/w or from about 50 to about 80% w/w.

The use of a plasticizer will often be desirable in order to improve the processibility of the coating. The plasticizer may also be a non-ionic surfactant, e.g. a non-ionic surfactant selected from the group consisting of diacetylated monoglycerides, diethylene glycol monostearate, ethylene glycol monostearate, glyceryl monooleate, glyceryl monostearate, propylene glycol monostearate, macrogol esters, macrogol stearate 400, macrogol stearate 2000, polyoxyethylene 50 stearate, macrogol ethers, cetomacrogol 1000, lauromacrogols, nonoxinols, octocinols, tyloxapol, poloxamers, polyvinyl alcohols, polysorbate 20, polysorbate 40, polysorbate 60, polysorbate 65, polysorbate 80, polysorbate 85, sorbitan monolaurate, sorbitan monooleate, sorbitan monopalmitate, sorbitan monostearate, sorbitan sesquioleate, sorbitan trioleate, sorbitan tristearate and sucrose esters; nitrobenzene, carbon disulfide, β-naphtyl salicylate, phthalyl glycolate, dioctyl phthalate.

Other suitable plasticizers appear from EP-B-0 746 310 to which reference is made.

### Preparation of a Pharmaceutical Composition

A pharmaceutical composition according to the invention may be prepared by extrusion, melt-extrusion, injection molding etc. The coating may be applied by co-extrusion of the coating with the composition, extrusion and dip coating, injection moulding and dip coating or by extrusion or injection moulding and solvent coating by spraying or dipping. A second fraction of the composition as well as any coating, if present, are preferably extrudable and/or injection mouldable.

### Material and Methods

The following materials have been employed:

Adeps Solidus, Unikem, pharmaceutical quality
Maize oil, Unikem, pharmaceutical quality
Coconut oil refined, Unikem, pharmaceutical quality
Arachis oil, Unikem, pharmaceutical quality
Cetyl stearyl alcohol (CSA), Brøste, pharmaceutical quality
Stearic acid, Sigma-Aldrich, analytical quality
Sodium lauryl sulphate (SDS), Unikem, pharmaceutical quality
PEO 100 000, DOW, pharmaceutical quality
PEO 200 000, DOW, pharmaceutical quality
PoloXamer188, BASF, pharmaceutical quality
Eudragit RL, Röhm, pharmaceutical quality
Calcitonin, Bachem, pharmaceutical quality
Hydrocortisone succinate, Sigma-Aldrich, analytical quality
Caffeine as base, Unikem, pharmaceutical quality

### Dissolution Testing

The dissolution test was carried out according to USP 25

Dissolution medium: Simulated Intestinal Fluid pH 6.8 according to USP 25 (prepared by dissolving 6.8 g monobasic potassium phosphate and 77 ml 0.2N NaOH in distilled water ad. 1 L and adjusting pH to pH 6.8)

### Apparatus

Two different apparatus were used:

Dissolution Apparatus 1: On-line system model SOTAX AT7 and UV detector Model PE lambda 2 using Disslab version 1.1 (the apparatus corresponds to USP 25 apparatus 2, paddle)

Test conditions were 37° C. and 50 rpm.

Dissolution Apparatus 2: Off-line system model VanKel bio-dis, Extended Release Tester with the standard VK 750D external heater/circulator for medium temperature control (the apparatus corresponds to USP 25, apparatus 3)

Test conditions were 37.5° C. and 12 dip per min

### Differentiall Scanning Calorimetry (DSC)

DSC model PE Pyris 1 and intercooler Model PE 1 with N₂ supply

### PE 50 μl pan

In general, a DSC curve was obtained using the following conditions:
1) Hold for 2.0 min at 10.00° C.
   Data points: 120
2) Heat from 10.00 to 50.00° C. at 15.00° C./min for Example 1 and 2 (Calcitonin)
   Data points: 266

### Flow Test

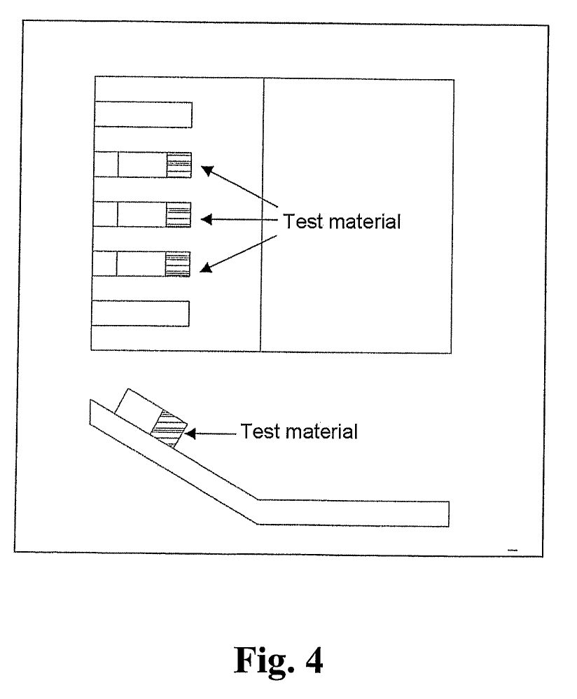

The flow-test apparatus is of stainless steel plate 2 mm thick, 135 mm long and 85 mm wide, bent across the long dimension, so that a portion (85×90 mm) lies flat and the other (85×45 mm) is positioned at a 45° angle. In upper surface of the inclined portion there are 7 furrows, 1 mm deep and 3 mm wide, starting at the higher edge. The furrows are aligned along the long dimension and there is a 5 mm distance between them. The first part of FIG. 4 schematically shows a flow-test apparatus from above, and the second part of FIG. 4 shows the flow-test apparatus from the side.

### Test Methods

The apparatus is placed in an oven at 38° C.±1° C. (the temperature is measured with a thermometer) for about ½ hour before use. The inner plugs are inserted into a clear (coat) tube (4 mm×12 mm) and inserted at one of the ends. The tubes are placed in the furrows, the plugs at the top. The apparatus with the tubes is placed in the oven 38° C. for 5 min. If the plug is melted (or sufficiently fluid) and flowed down to the end of the tube or out of it, the formulation passes the test.

### EXAMPLES

### Example 1

### A Composition According to the Invention Containing 50 000 Units Calcitonin Oil Dosage Unit

A three-layered coated composition having a shape as outlined in FIG. 1 containing Calcitonin as active substance was prepared as follows:

The outer plugs were prepared in accordance with the description in EP 0493 513 B1) using

| | % w/w |
|---|---|
| PEO 200 000 | 60 |
| Citric Acid | 5 |
| PEG 2000 ms | 5 |
| Maize starch | 30 |

The inner plug containing calcitonin had the following composition

| | % w/w | mg |
|---|---|---|
| Adeps solidus | 63 | 630 |
| Maize oil | 27 | 270 |
| Calcitonin | 10 | 100 |

The oily ingredients are heated in a water bath until melted keeping the temperature below 40° C., then the melt is poured into a mortar and cooled to about 27° C. under constant stirring. Calcitonin is added to the melt and stirred to a homogeneous mass to obtain the inner matrix composition.

For production of inner plugs, a mould (a plate with 16 round holes, 4 mm in diameter×4 mm in length for a 12 mm dosage unit) is used. The mould is placed in a refrigerator for a least one hour before the preparation and then the cold mould is placed on a glass plate and the molten/softened inner matrix composition is poured on the mould and, if necessary, pressed into the holes of the mould. Cold compression (i.e. partially melt) is used to avoid sedimentation of the active substance. The mould was cooled, wrapped in film and stored in the refrigerator until use. The inner plugs are weighted to ensure the uniformity of mass before use.

The matrix was coated with a composition containing

| | % w/w |
|---|---|
| Ethylcellulose | 79 |
| Cetyl stearyl alcohol | 20 |
| Titandioxide | 1 |

using the procedure described in EP 0493 513 B1

The inner and outer plugs were assembled by pressing the cold inner plug into the coat with a cold metal pin. The outer plugs are assembled in the same manner. The finished coated composition is stored in the refrigerator until use

The coat was 4 mm in diameter, 12 mm long and 0.8 mm thick. The outer plugs were 4 mm in diameter and 4 mm long. The inner plug was 4 mm in diameter and 4 mm long.

The composition was subjected to dissolution testing over a period of 5 h using Dissolution Apparatus 1 and the dissolutions curve was obtained at 37° C. and 50 rpm using UV detection at 273 nm.

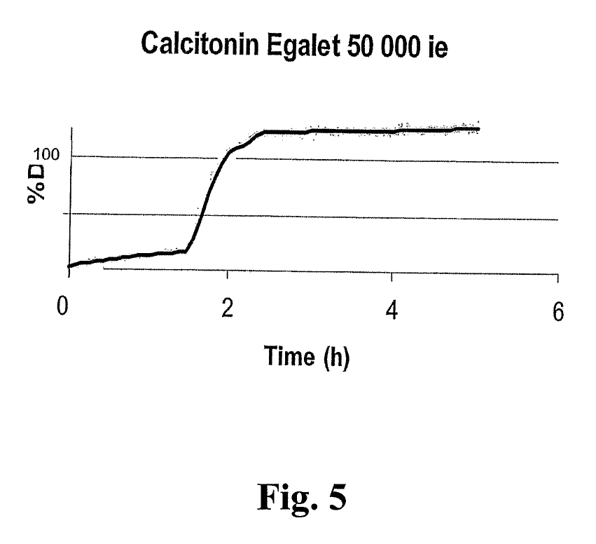

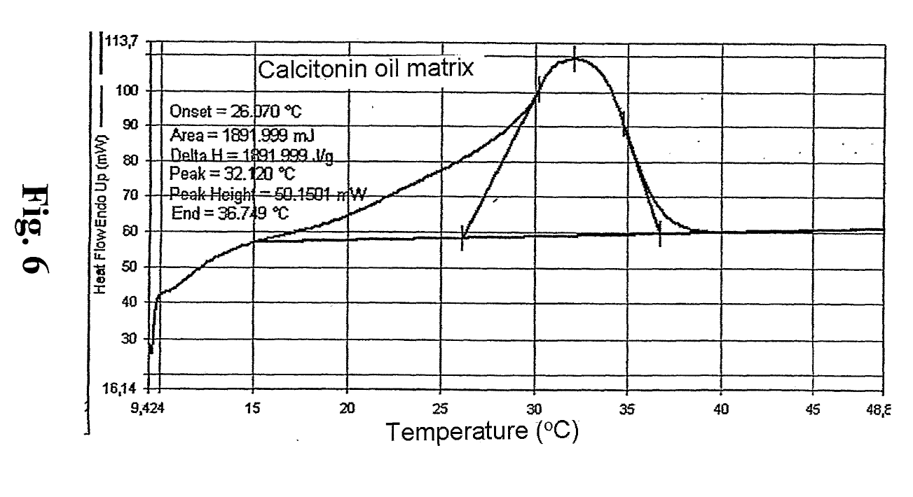

The result is given in FIG. 5. After a lag time of about 1½-2¼ h, the release of calcitonin starts and all Calcitonin has been released after about ½ h.

The composition was also subjected to DSC using the following conditions
1) Hold for 2.0 min at 10.00° C.
   Data points: 120
2) Heat from 10.00 to 50.00° C. at 15.00° C./min
   Data points: 266

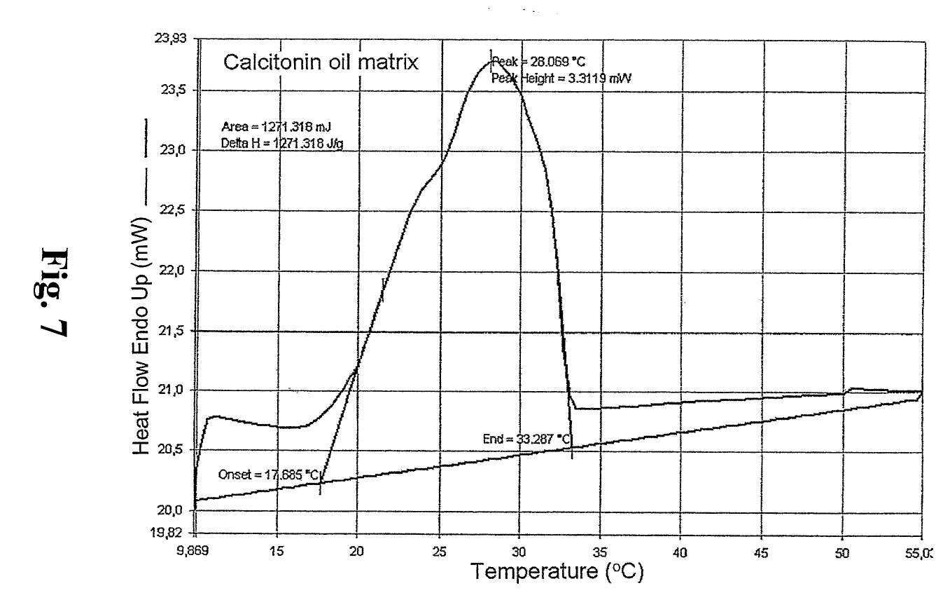

FIG. 6 illustrates the DSC profile of Calcitonin oil matrix

The Calcitonin formulation has a peak temp. 32.1° C. and an end point temperature of the melting interval at 36.7° C., which is below or at body temperature.

### Example 2

### A Composition According to the Invention Containing 15,000 Units Calcitonin

The composition was prepared as described in Example 1 above, but the inner plug had a different composition:

| Material | % w/w | mg |
|---|---|---|
| Adeps solidus | 66.8 | 2004 |
| Coconut oil | 28.8 | 864 |
| Calcitonin | 4.4 | 132 |

The composition was subjected to dissolution testing using Dissolution apparatus 2. After about 5 h start the release of the Calcitonin in simulated Intestinal Fluid (the solution change from a clear colourless solution to a cloudy solution at the end of release) and end with in ½ h.

The dosage unit has burst.

The composition was also subjected to DSC using the following conditions:
1) Hold for 2.0 min at 10.00° C.
   Data points: 120
2) Hear from 10.00 to 50.00° C. at 15.00° C./min
   Data points: 266

FIG. 7 illustrates the DSC profile of calcitonin oil matrix

### Example 3

### Composition According to the Invention Containing 20 mg Hydrocortisone as an Active Substance

The composition was prepared as described in Example 1 above, but the inner plug had a different composition:

| Material | % w/w | mg |
|---|---|---|
| Adeps solidus | 44.8 | 672 |
| Maize oil | 19.2 | 288 |
| Hydrocortisone succinate | 36 | 540 |

The oily ingredients was heated in a water bath until melted keeping the temperature below 40° C., the melt poured into a mortar, cooled to about 27° C. under constant stirring, hydrocortisone succinate was added to the melt and stirred to an homogeneous mass.

The composition was subjected to dissolution testing over a period of 15 h using Dissolution Apparatus 1 and the dissolutions curve was obtained at 37° C. and 50 rpm using UV detection at 273 nm.

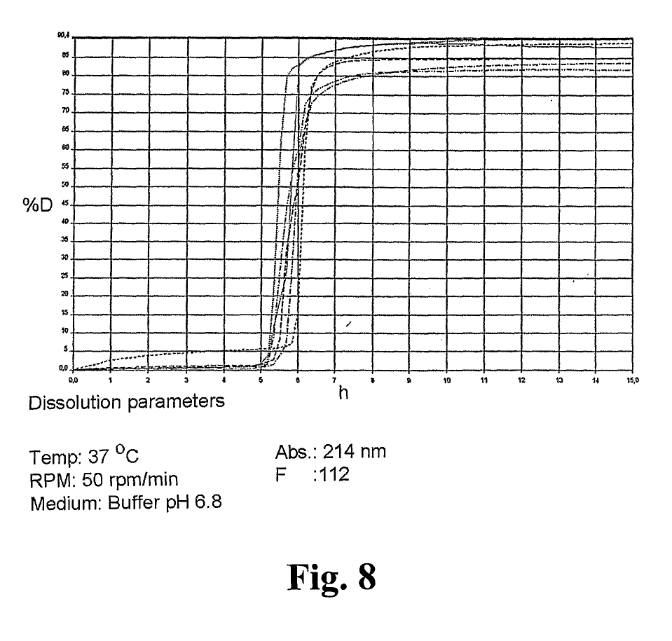

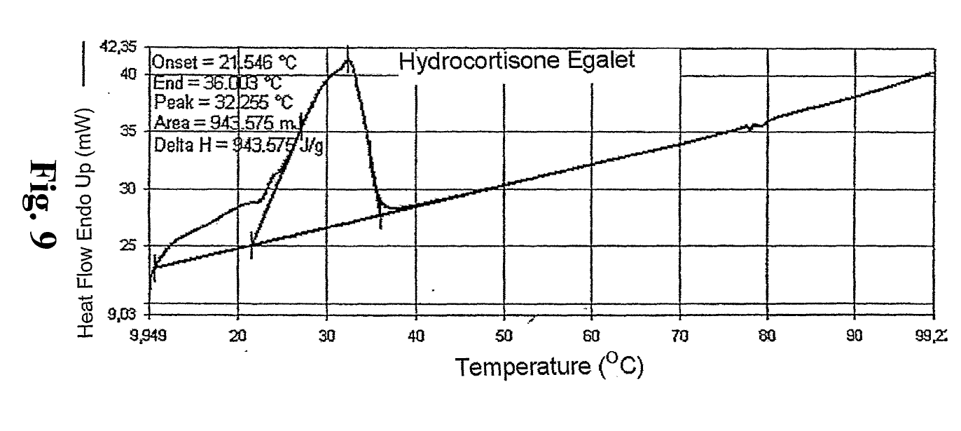

The result is given in FIG. 8. After 5½ h start the release of the hydrocortisone succinate and end after ½ h. The dosage unit has burst.

The composition was also subjected to DSC using the following conditions:
1) Hold for 2.0 min at 10.00° C.
   Data points: 120
2) Heat from 10.00 to 100.00° C. at 10.00° C./min
   Data points:

FIG. 9 illustrates the DSC profile of the hydrocortisone succinate composition

Hydrocortisone succinate composition has a peak temperature at 32.3° C. and an end temperature at 36.0° C., which is below or at body temperature.

### Example 4

### Composition According to the Invention Containing 1.5 mg Caffeine

The composition was prepared as described in Example 1 above, but the inner plug had a different composition:

| Material | % w/w |
|---|---|
| Adeps solidus | 67.9 |
| Maize oil | 29.1 |
| Caffeine | 3 |

The oily ingredients were heated on the hot plate until melted but not above 40° C., the melt was poured into a mortar, cooled to about 27° C. under constant stirring and then caffeine was added to the melt and stirred to a homogeneous mass.

The tablet is assembled by pressing the cold inner plugs into the shell, with a cold metal stick or by using the glass plate as under layer for the inner plug. The outer plugs are assembled in the same manner. The finished tablet is stored in the refrigerator until use.

The composition was subjected to dissolution testing over a period of 15 h using Dissolution Apparatus 1 and the dissolutions curve was obtained at 37° C. and 50 rpm using UV detection at 273 nm.

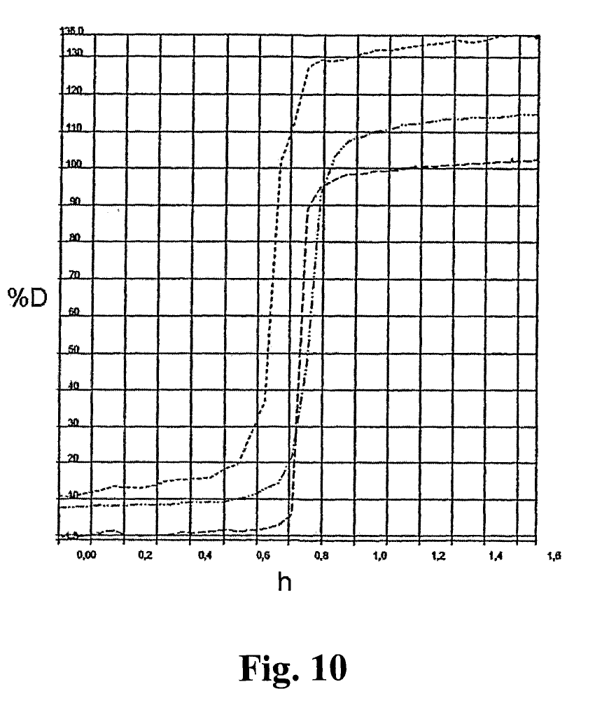

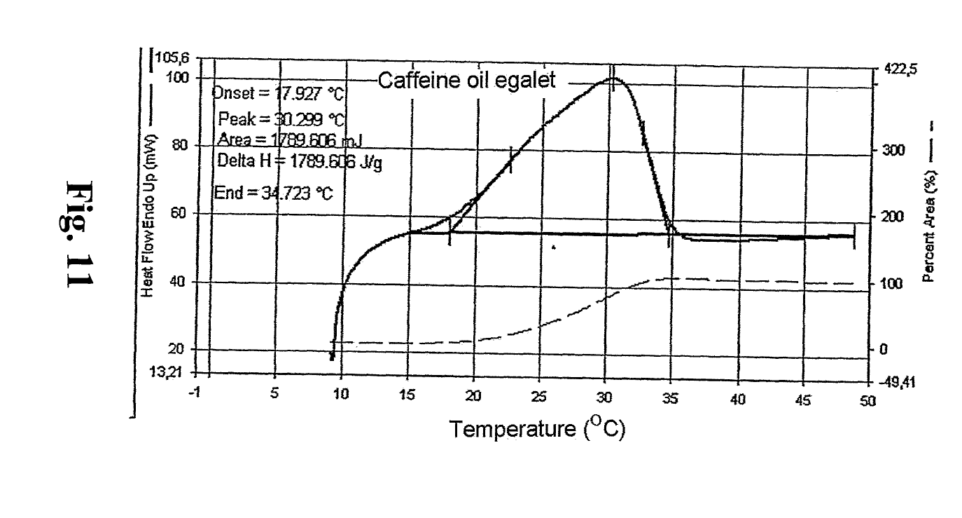

The result is given in FIG. 10. After 1.5 h start the release of the caffeine and end after ½ h. The dosage unit has burst after 1.5 h.

The composition was also subjected to DSC using the following conditions:
1) Hold for 2.0 min at 10.00° C.
   Data points: 120
2) Heat from 10.00 to 50.00° C. at 15.00° C./min
   Data points: 266

FIG. 11 illustrates the DSC profile of caffeine composition.

The caffeine composition has a peak temperature at 30.3° C. and an end temperature at 34.7° C., which is below body temperature.

### Example 5

### Compositions According to the Invention

The following compositions (Table 1) are examples on dispersion media that can be used to prepare the inner plug of a composition according to the invention. The resulting inner plug is an oil-based matrix with a suitable melting point off set (as defined herein) to enable that the inner plug can flow out of the coating or the remaining part of the coating once the outer plugs have disappeared.

Adding SDS (sodium lauryl sulfate), lower the tension of the oil droplet.

The inner plug is used to achieve a faster release of the active ingredient. In particular for water-soluble active ingredients a faster release will be achieved.

TABLE 1

| | Matrix | | Comments (mp are given |
|---|---|---|---|
| Batch No. | Ingredient | % w/w | as onset melting points) |
| 20020314-1 | Adeps solidus | 90% | Mp. 44° C. |
| | Cetyl stearyl alchol | 10% | |
| 20020314-2 | Adeps solidus | 80% | Mp. 53° C. |
| | Cetyl stearyl alchol | 20% | |
| 20020314-3 | Adeps solidus | 70% | Mp. 50° C. |
| | Cetyl stearyl alchol | 30% | |
| 20020318-1 | Adeps solidus | 97 | Onset: 35.2° C. |
| | Stearic acid | 3 | Peak: 40.3° C. |
| 20020318-2 | Adeps solidus | 90 | Onset: 29.7° C. |
| | Stearic acid | 10 | Peak: 34.8° C. |
| 20020318-3 | Adeps solidus | 70 | Onset: 30.8° C. |
| | Stearic acid | 30 | Peak: 34.7° C. |
| 20020321-1 | Adeps solidus | 90 | Onset: 35° C. |
| | Arachis oil | 10 | Peak: 38° C. |
| 20020321-2 | Adeps solidus | 80 | Onset: 27° C. |
| | Arachis oil | 20 | Peak: 34° C. |
| 20020321-3 | Adeps solidus | 70 | Onset: 27° C. |
| | Arachis oil | 30 | Peak: 34° C. |
| | Adeps solidus | 70 | |
| | Arachis oil | 30 | |
| | Na lauryl sulphate | | |
| | (SDS) | | |

### Example 6

### Various Coatings for Compositions According to the Invention

The following compositions (Table 2) are examples of different coats that may be applied on pharmaceutical compositions according to the invention. This could for example result in three-layered coated compositions having a shape as outlined in FIG. 2, comprising an inner plug, two outer plugs and a coat. The coat compositions in table 2 can e.g. be applied on the dosage unit described in example 1.

TABLE 2

| Batch No. | Ingredient | % w/w |
|---|---|---|
| 05-0131-058 | PEO 100 000 | 98 |
| | CSA | 2 |
| 06-0016-058 | PEO 100 000 | 85 |
| | CSA | 15 |
| 06-0002-058 | PEO 100 000 | 70 |
| | CSA | 30 |
| 06-0003-058 | PoloXamer188 | 50 |
| | Eudragit RL | 50 |
| 060004-058 | Eudragit RL | 70 |
| | PoloXamer188 | 30 |

1. A coated solid pharmaceutical composition in the form of a single dosage unit for oral use, the composition comprising a first and a second fraction,
i) the first fraction comprises a therapeutically and/or prophylactically active substance dispersed in a dispersion medium that is sufficiently fluid at body temperature,
wherein the first fraction is placed between two second fractions, between a coating and a second fraction, or alternatively is completely surrounded by a second fraction, the first fraction has a melting point cut off of at the most about 38° C., and
the active substances contained in the first fraction is released relatively fast once the second fraction has eroded, and
ii) the second fraction comprises an erodable matrix comprising a substantially water soluble or crystalline polymer or a mixture of substantially water soluble and/or crystalline polymers, the polymer being a polyethylene oxide having a MW of from 20,000 daltons to about 700,000 or a block copolymer of ethylene oxide and propylene oxide having a MW of from about 3,000 to about 30,000 daltons, or a mixture thereof,
wherein the composition is coated with a coating having at least one opening exposing one surface of the matrix of the second fraction, and the coating is substantially insoluble in and impermeable to body fluids.

2. A composition according to claim 1, wherein the dispersion medium of the first fraction—when tested according to the Flow Test described herein—passes the test.

3. A composition according to claim 1, wherein the first fraction—when tested according to the Flow Test described herein—passes the test.

4. A composition according to claim 1, wherein the dispersion medium of the first fraction has a melting point cut off of at the most about 50° C.

5. A composition according to claim 1, wherein the dispersion medium of the first fraction has a melting point onset of about 0° C. or more such as, e.g. about 5° C. or more, about 10° C. or more, about 15° C. or more, about 20° C. or more or about 25° C. or more.

6. A composition according to claim 1, wherein the first fraction has a melting point onset of about 0° C. or more such as, e.g. about 5° C. or more, about 10° C. or more, about 15° C. or more, about 20° C. or more or about 25° C. or more.

7. A composition according to claim 1 having a delayed release of the active substance contained in the first fraction.

8. A composition according to claim 7, wherein—when tested in an in vitro dissolution test—at the most about 5% w/w of the active substance contained in the first fraction is released from the composition within 15 min or more after start of the test.

9. A composition according to claim 8, wherein at the most 5% w/w of the active substance contained in the first fraction is released from the composition within 30 min or more such as, e.g., 1 h or more, 1.5 h or more, 2 h or more, 3 h or more, 4 h or more, 5 h or more, 6 h or more, 7 hours or more or 8 hours or more after start of the test.

10. A composition according to claim 1, wherein the dispersion medium comprises one or more solvents, one or more co-solvents, one or more oils, one or more waxes and/or one or more semi-solid materials.

11. A composition according to claim 1, wherein the dispersion medium is a lipid-based medium.

12. A composition according to claim 1, wherein the dispersion medium is an aqueous-based medium.

13. A composition according to claim 10, wherein the dispersion medium comprises a substance selected from the group consisting of cocoa butter, coca butte substitutes like e.g. vegetable oils modified by esterification, hydrogenation, fractionation etc., shea butter, adeps solidus including those using different triglycerides as starting materials, waxes including beeswax, theobroma oil, hydrogenated vegetable oil bases such as fattybase, wecobee bases, witepsol based water-soluble bases vegetable oil including coconut oil, palm kern oil, cotton seed oil, olive oil, maize oil, peanut oil, sesame oil, sunflower oil, and miglyol 813, and mixtures thereof.

14. A composition according to claim 1, wherein the dispersion medium comprises a polyethylene glycol that has a molecular weight of 20,000 or less, glycerinated gelatine, fatty acid esters of polyethylene glycol.

15. A composition according to claim 12, medium in the first fraction is aqueous based and comprises surface-active agents, emulsifiers and/or nano particles.

16. A composition according to claim 1, wherein the first fraction is contained in an inner layer of the composition, at least one surface of the inner layer being in contact with at least one surface of the matrix of the second fraction.

17. A composition according to claim 1, wherein the release of the active substance from the first fraction follows a kinetic that is different from a zero order release.

18. A composition according to claim 1, wherein the release of the active substance from the first fraction—when tested using an in vitro dissolution test method—is at the most about 20% w/w such as at the most about 15% w/w, at the most about 10% w/w, at the most 5% w/w, at the most about 2.5% w/w, at the most about 1% w/w or at the most about 0.1% w/w when measured 2 hours or more such as, e.g., 3 hours or more, 4 hours or more, 5 hours or more, 6 hours or more, 7 hours or more, 8 hours or more, 9 hours or more, 10 hours or more, 11 hours or more, 12 hours or more, 13 hours or more, 14 hours or more, 15 hours or more or 16 hours or more after start of the test.

19. A composition according to claim 1, wherein at least about 75% w/w such as, e.g., at least about 80% w/w, at least about 85% w/w, at least about 90% w/w or at least about 95% w/w of the total amount of the active substance contained in the first fraction is released within 90 minutes—when tested using an in vitro dissolution test method and the starting point of the test being defined as the point in time when 20% w/w of the total amount of the active substance contained in the first fraction is released.

20. A composition according to claim 1, wherein the amount of the active substance in the first fraction corresponds to a daily or part of a daily therapeutic dose.

21. A composition according to claim 1 further comprising an active substance in the second fraction.

22. A composition according to claim 20, wherein the active substance in the second fraction is the same or different from the active substance contained in the first fraction.

23. A composition according to claim 1, wherein the active substance has a water-solubility at room temperature of at the most about 3 mg/ml.

24. A composition according to claim 1, wherein the active substance is a peptide, a polypeptide or a protein.

25. A composition according to claim 1, wherein the polyethylene glycol, a polyethylene oxide and/or a block copolymer of ethylene oxide and propylene oxide has a molecular weight of from about 20,000 to about 600,000 daltons, from about 35,000 to about 500,000 daltons, from about 35,000 to about 400,000 daltons, from about 35,000 to about 300,000 daltons, from about 50,000 to about 300,000 daltons, such as, e.g. about 35,000 daltons, about 50,000 daltons, about 75,000 daltons, about 100,000 daltons, about 150,000 daltons, about 200,000 daltons, about 250,000 daltons, about 300,000 daltons or about 400,000 daltons.

26. A composition according to claim 25, wherein the block copolymer of ethylene oxide and propylene oxide comprises up to about 30% w/w of the propylene oxide based block, and has a molecular weight of about 5,000 to about 30,000 daltons such as, e.g. from about 8,000 to about 15,000 daltons.

27. A composition according to claim 1, wherein the matrix of the second fraction comprises a polymer which has a melting point of about 20-120° C. such as, e.g. from about 30 to about 100° C. or from about 40 to about 80° C.

28. A composition according to claim 1, wherein the coating is a coating having at least one opening exposing at the one surface of said matrix of the second fraction, the coating comprising
a) a first cellulose derivative which has thermoplastic properties and which is substantially insoluble in the aqueous medium in which the composition is to be used,
and at least one of
b) a second cellulose derivative which is soluble or dispersible in water,
c) a plasticizer, and
d) a filler.

29. A composition according to claim 28, wherein in the aqueous medium in which the composition is to be used, the coating does not completely crumble or erode before the matrix of the second fraction has completely eroded.

30. A composition according to claim 28, wherein said first cellulose derivative is a cellulose ether which, when heated, is shapeable by molding or extrusion, including injection molding, blow molding and compression molding.

31. A composition according to claim 30 in which the cellulose ether comprises at least one ethylcellulose.

32. A composition according to claim 31 in which said ethylcellulose has an ethoxyl content in the range of 44.5-52.5%.

33. A composition according to claim 32 in which said ethylcellulose has an ethoxyl content in the range of 45-49.5%.

34. A composition according to claim 28 in which said first cellulose derivative is selected from the group consisting of cellulose acetate, cellulose propionate and cellulose nitrate.

35. A composition according to claim 28 in which said second cellulose derivative is selected from the group consisting of methylcellulose, carboxymethylcellulose and salts thereof, cellulose acetate phthalate, microcrystalline cellulose, ethylhydroxyethylcellulose, ethylmethylcellulose, hydrocyethylcellylose, hydroxyethylmethylcellulose, hydroxypropylcellulose, hydroxymethylcellulose and hydroxymethylpropylcellulose.

36. A composition according to claim 35 in which said salt of carboxymethylcellulose is selected from the group consisting of alkali metal and alkaline earth metal salts.

37. A composition according to claim 28, in which said plasticizer is selected from the group consisting of phosphate esters; phthalate esters; amides; mineral oils; fatty acids and esters thereof with polyethylene glycol, glycerin or sugars; fatty alcohols and ethers thereof with polyethylene glycol, glycerin or sugars; vegetable oils and hydrogenated vegetable oils; nitrobenzene, carbon disulfide, β-naphtyl salicylate, phthalyl glycolate, and diocyl phthalate.

38. A composition according to claim 37 in which said fatty alcohol is selected from the group consisting of cetostearyl alcohol, cetyl alcohol, stearyl alcohol, oleyl alcohol and myristyl alcohol.

39. A composition according to claim 28 in which said plasticizer is a non-ionic surfactant.

40. A composition according to claim 28, wherein said filler is a filler, a diluent, a binder, a lubricant a disintegrant or a water-soluble anti-oxidant, a lipid soluble antioxidant and/or a preservative.
